(19) United States
(12) Patent Application Publication
(10) Pub. No.: US 2007/0154500 A1
(43) Pub. Date: Jul. 5, 2007

(54) COMPOSITION CONTAINING CONCAVE PARTICLES AND A DISPERSANT, PROCESSES AND USES

(75) Inventors: Guillaume Cassin, Villebon Sur Yvette (FR); Sylvie Poret Fristot, Rungis (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/614,202

(22) Filed: Dec. 21, 2006

Related U.S. Application Data
(60) Provisional application No. 60/814,889, filed on Jun. 20, 2006.

(30) Foreign Application Priority Data
Dec. 21, 2005 (FR) 05 13091

(51) Int. Cl. A61K 8/89; A61K 8/81; A61K 8/92

(52) U.S. Cl. 424/401; 424/70.12; 424/70.7

(57) ABSTRACT

The present invention relates to a composition containing concave particles, at least one dispersant for these concave particles, and optionally porous particles. This composition is nonpulverulent. The present invention also relates to a treatment process employing this composition and to the uses of this composition, in particular for conferring a matt appearance on keratinous substances and/or for softening imperfections of the relief of the skin and/or for concealing microrelief features, wrinkles, fine lines and pores of the skin.

### REFERENCE TO PRIOR APPLICATIONS

This application claims priority to U.S. provisional application No. 60/814,889 filed Jun. 20, 2006, and to French invention 0513091 filed Dec. 21, 2005, both incorporated herein by reference.

### FIELD OF THE INVENTION

The present invention relates to novel compositions useful for treating, caring for or making up human keratinous substances, such as the skin, in particular to compositions intended to mattify human keratinous substances. The compositions according to the present invention comprise concave particles and at least one dispersant for these concave particles.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Mattifying compositions are compositions which prevent the support on which they are applied from shining. Applied to the face, a mattifying composition prevents the skin from shining and gives a uniform complexion. Compositions for treating or caring for the skin which exhibit mattifying properties are generally used to solve the problems of shininess brought about by an excess of seburn. Mattifying makeup compositions aim to improve the makeup base and thus to improve the hold of the makeup. Mattifying compositions give a matt appearance to the skin due to the scattering of light at the surface of the skin. These compositions can also be used to soften defects of the skin such as microrelief features, wrinkles, fine lines, pores or variations in colour.

Generally, mattifying cosmetic compositions are formulated using inorganic fillers (ZnO, SiO2, . . . ), which may or may not be coated, and starch. These particles are well known and widely employed but exhibit the disadvantage of settling out and are characterized by a rather unpleasant rough feel.

Mattifying compositions based on aqueous styrene/acrylic dispersions (FR 2 801 215), on aqueous polytetrafluoroethylene dispersions (FR 2 820 977) and on cellulose beads with a diameter of less than 10 μm (FR 2 846 878) are also known.

The problem posed in the present invention is that of obtaining cosmetic compositions exhibiting satisfactory mattifying properties even when they comprise a large amount of mattifying fillers.

This is because, when a mattifying composition is prepared, it is often difficult to introduce the mattifying fillers without the latter agglomerating. In point of fact, the appearance of agglomerates has the consequence of resulting in a nonhomogeneous composition having unsatisfactory mattifying properties.

In addition, mattifying compositions comprising a large amount of fillers generally exhibit an excessively great dusty and powdered effect.

### SUMMARY OF THE INVENTION

Advantageously and unexpectedly, the inventors have now discovered that it is possible to obtain compositions exhibiting satisfactory mattifying properties by combining concave particles and at least one dispersant for these concave particles.

The compositions according to the present invention comprise homogeneously dispersed concave particles. Agglomerates of concave particles are generally not observed during the preparation of these compositions; it is thus possible to obtain mattifying compositions comprising high concentrations of concave particles.

Furthermore, the compositions according to the present invention exhibit satisfactory mattifying properties even when they comprise low concentrations of concave particles. Consequently, the compositions according to the present invention are also advantageous economically.

In addition, the organoleptic characteristics of these compositions, and in particular their feel, are also satisfactory.

Other characteristics, aspects, objects and advantages of the present invention will become even more clearly apparent by reading the description and the examples which follow.

### BRIEF DESCRIPTION OF THE DRAWING

FIG. 1 shows a concave particle formed of portions of spheres with the shape of a bowl.

### DETAILED DESCRIPTION OF THE PREFERRED EMBODIMENTS

A preferred aspect of the present invention is a composition comprising concave particles and at least one dispersant for these concave particles. Preferably, the composition is a nonpulverulent cosmetic composition. As used herein, the term non pulverulent means non powdery.

Within the meaning of the present invention, the “dispersant for concave particles” is a compound which, when it is introduced in a content of 2% by weight with respect to the total weight of the composition, under standard temperature and pressure conditions, into an oil-in-water emulsion comprising 1.00% by weight of ammonium polyacryldimethyltauramide, 5.0% by weight of cyclohexasiloxane, 1.70% by weight of glycerol, 0.30% by weight of stearyl alcohol, 0.70% by weight of glyceryl stearate/PEG 100 stearate, 0.50% by weight of dimyristyl tartrate/cetearyl alcohol/C12-15 pareth-7/PPG25 laureth-25, 1.5% by weight of concave particles and 0.5% by weight of preservatives (the remainder to 100% by weight being water) with respect to the total weight of the composition, is dissolved and makes it possible to reduce the R value, which is the ratio of the specular reflection to the diffuse reflection, of this composition; preferably it makes it possible to reduce the R value to a value of less than or equal to 1.

The R value defines the mattifying nature of the compositions; it is defined below.

The mattifying nature of the compositions according to the present invention is defined by a measurement with a gonioreflectometer. For this, the composition is spread over a contrast card (Prufkarte type 24/5-250 cm² sold by Erichsen) using a mechanical film drawer (wet thickness 30 microns). The composition is subsequently dried overnight at a temperature of 37° C. and then the reflection is measured using a gonioreflectometer. The result obtained is the ratio R of the specular reflection to the diffuse reflection. The R value decreases as the mattifying effect increases. The cosmetic compositions according to the invention are those which give an R value of less than 1, preferably of less than 0.75.

The compositions according to the present invention preferably reveal the grain of the skin while masking the imperfections thereof; more generally, they make it possible to correct dyschromias of the skin.

In particular, the present invention is targeted at a cosmetic composition, preferably a nonpulverulent cosmetic composition, wherein it comprises, in a physiologically acceptable medium, concave particles and at least one compound chosen from vinylpyrrolidone homopolymers and polyalkylene glycols with a molecular weight of less than 20 000 g/mol.

The present invention is also targeted at a process for cosmetically treating, caring for or making up keratinous substances, in particular the skin, which comprises applying, to the keratinous substances, the composition according to the present invention; in particular, it is targeted at a cosmetic treatment process, that is to say a nontherapeutic treatment process, intended to confer, on keratinous substances, a matt appearance and/or to soften imperfections of the relief of a skin and/or to conceal microrelief features, wrinkles, fine lines and pores of the skin.

The present invention is also targeted at the cosmetic use of the composition according to the present invention for conferring a matt appearance on keratinous substances and/or for softening imperfections of the relief of the skin and/or for concealing microrelief features, wrinkles, fine lines and pores of the skin.

The composition according to the invention comprises concave particles. These particles thus have a surface exhibiting a rounded interior.

The concave particles are preferably portions of hollow spheres composed of a material, in particular an organosilicone material.

The concave particles advantageously have a mean diameter ranging from 0.05 μm to 20 μm.

The portions of hollow spheres used in the composition according to the invention can have the shape of truncated hollow spheres exhibiting a single orifice communicating with their central cavity and having a transverse cross section with the shape of a horseshoe or arch. They are in particular hollow half-spheres.

The organosilicone material is preferably a crosslinked polysiloxane with a three-dimensional structure; it preferably comprises, indeed even is composed of, units of formula (I): SiO₂, and of formula (II): R¹SiO_{1.5},

in which R1 denotes an organic group having a carbon atom directly connected to the silicon atom. The organic group can be a reactive organic group or an unreactive organic group; preferably it is an unreactive organic group.

The unreactive organic group can be a C₁-C₄ alkyl group, in particular a methyl, ethyl, propyl or butyl group, or a phenyl group; preferably it is a methyl group.

The reactive organic group can be an epoxy group, a (meth)acryloyloxy group, an alkenyl group, a mercaptoalkyl group, an aminoalkyl group or a haloalkyl group, a glyceroxy group, a ureido group or a cyano group. Preferably, the reactive organic group is an epoxy group, a (meth)acryloyloxy group, an alkenyl group, a mercaptoalkyl group or an aminoalkyl group. When the reactive organic group comprises an alkyl radical, this alkyl radical generally comprises from 2 to 6 carbon atoms, in particular from 2 to 4 carbon atoms.

Mention may especially be made, as epoxy group, of a 2-glycidoxyethyl group, a 3-glycidoxypropyl group or a 2-(3,4-epoxycyclohexyl)propyl group.

Mention may especially be made, as (meth)acryloyloxy group, of a 3-methacryloyloxypropyl group or a 3-acryloyloxypropyl group.

Mention may especially be made, as alkenyl group, of a vinyl, allyl or isopropenyl group.

Mention may especially be made, as mercaptoalkyl group, of a mercaptopropyl or mercaptoethyl group.

Mention may especially be made, as aminoalkyl group, of a 3-[(2-aminoethyl)amino]propyl group, a 3-aminopropyl group or an N,N-dimethylaminopropyl group.

Mention may especially be made, as haloalkyl group, of a 3-chloropropyl group or a trifluoropropyl group.

Mention may especially be made, as glyceroxy group, of a 3-glyceroxypropyl group or a 2-glyceroxyethyl group.

Mention may especially be made, as ureido group, of a 2-ureidoethyl group.

Mention may especially be made, as cyano group, of a cyanopropyl or cyanoethyl group.

Preferably, in the unit of formula (II), R¹ denotes a methyl group.

Advantageously, the organosilicone material comprises the units (I) and (II) according to a unit (I)/unit (II) molar ratio ranging from 30/70 to 50/50, preferably ranging from 35/65 to 45/55.

The particles of organosilicone material can be obtained according to the process described below comprising:

(a) the introduction into an aqueous medium, in the presence of at least one hydrolysis catalyst and optionally of at least one surfactant, of a compound (III) of formula SiX₄ and of a compound (IV) of formula RSiY₃, where X and Y denote, independently of one another, a C₁-C₄ alkoxy group, an alkoxyethoxy group including a C₁-C₄ alkoxy group, a C₂-C₄ acyloxy group, an N,N-dialkylamino group including a C₁-C₄ alkyl group, a hydroxyl group, a halogen atom or a hydrogen atom and R denotes an organic group comprising a carbon atom connected directly to the silicon atom; the R group corresponding to the R¹ group defined above; and

(b) the operation in which the mixture resulting from stage (a) is brought into contact with an aqueous solution including at least one polymerization catalyst and optionally at least one surfactant, at a temperature of between 30 and 85° C., for at least two hours.

Stage (a) corresponds to a hydrolysis reaction and stage (b) corresponds to a condensation reaction.

In stage (a), the molar ratio of the compound (III) to the compound (IV) generally ranges from 30/70 to 50/50, advantageously from 35/65 to 45/55, and is preferably 40/60.

The ratio by weight of the water to the total of the compounds (III) and (IV) preferably ranges from 10/90 to 70/30. The order of introduction of the compounds (III) and (IV) generally depends on their rate of hydrolysis. The temperature of the hydrolysis reaction generally ranges from 0 to 40° C. and usually does not exceed 30° C. in order to prevent premature condensation of the compounds.

For the X and Y groups of the compounds (III) and (IV):

Mention may especially be made, as C₁-C₄ alkoxy group, of the methoxy or ethoxy groups;

Mention may especially be made, as alkoxyethoxy group including a C₁-C₄ alkoxy group, of the methoxyethoxy or butoxyethoxy groups;

Mention may especially be made, as C₂-C₄ acyloxy group, of the acetoxy or propionyloxy groups;

Mention may especially be made, as N,N-dialkylamino group including a C₁-C₄ alkyl group, of the dimethylamino or diethylamino groups;

Mention may especially be made, as halogen atom, of the chlorine or bromine atoms;

Mention may especially be made, as compounds of formula (III), of tetramethoxysilane, tetraethoxysilane, tetrabutoxysilane, trimethoxyethoxysilane, tributoxyethoxysilane, tetraacetoxysilane, tetrapropionyloxysilane, tetra(dimethylamino)silane, tetra(diethylamino)silane, silanetetraol, chlorosilanetriol, dichlorodisilanol, tetrachlorosilane or chlorotrihydrosilane. Preferably, the compound of formula (III) is chosen from tetramethoxysilane, tetraethoxysilane, tetrabutoxysilane, and their mixtures. More preferably, it is tetraethoxysilane.

The compound of formula (III) results, after the polymerization reaction, in the formation of the units of formula (I).

The compound of formula (IV) results, after the polymerization reaction, in the formation of the units of formula (II).

The R group in the compound of formula (IV) has the meaning as described for the R¹ group for the compound of formula (II).

Mention may especially be made, as examples of compounds of formula (IV) comprising an unreactive organic R group, of methyltrimethoxysilane, ethyltriethoxysilane, propyltributoxysilane, butyltributoxysilane, phenyltrimethoxyethoxysilane, methyltributoxyethoxysilane, methyltriacetoxysilane, methyltripropionyloxysilane, methyltri(dimethylamino)silane, methyltri(diethylamino)silane, methylsilanetriol, methylchlorodisilanol, methyltrichlorosilane or methyltrihydrosilane.

Mention may especially be made, as examples of compounds of formula (IV) comprising a reactive organic R group, of:

silanes having an epoxy group, such as (3-glycidoxypropyl)trimethoxysilane, (3-glycidoxypropyl)triethoxysilane, [2-(3,4-epoxycyclohexyl)ethyl]trimethoxysilane, (3-glycidoxypropyl)methyldimethoxysilane, (2-glycidoxyethyl)methyldimethoxysilane, (3-glycidoxypropyl)dimethylmethoxysilane or (2-glycidoxyethyl)dimethylmethoxysilane;

silanes having a (meth)acryloxy group, such as (3-methacryloxypropyl)trimethoxysilane or (3-acryloxypropyl)trimethoxysilane;

silanes having an alkenyl group, such as vinyltrimethoxysilane, allyltrimethoxysilane or isopropenyltrimethoxysilane;

silanes having a mercapto group, such as mercaptopropyltrimethoxysilane or mercaptoethyltrimethoxysilane;

silanes having an aminoalkyl group, such as (3-aminopropyl)trimethoxysilane, (3-[(2-aminoethyl)amino]propyl)trimethoxysilane, (N,N-dimethylaminopropyl)trimethoxysilane or (N,N-dimethylaminoethyl)trimethoxysilane;

silanes having a haloalkyl group, such as (3-chloropropyl)trimethoxysilane or trifluoropropyltrimethoxysilane;

silanes having a glyceroxy group, such as (3-glyceroxypropyl)trimethoxysilane or di(3-glyceroxypropyl)dimethoxysilane;

silanes having a ureido group, such as (3-ureidopropyl)trimethoxysilane, (3-ureidopropyl)methyldimethoxysilane or (3-ureidopropyl)dimethylmethoxysilane;

silanes having a cyano group, such as cyanopropyltrimethoxysilane, cyanopropylmethyldimethoxysilane or cyanopropyldimethylmethoxysilane.

Preferably, the compound of formula (IV) comprising a reactive organic R group is chosen from silanes having an epoxy group, silanes having a (meth)acryloyloxy group, silanes having an alkenyl group, silanes having a mercapto group or silanes having an aminoalkyl group.

Examples of compounds (III) and (IV) which are preferred for the implementation of this invention are respectively tetraethoxysilane and methyltrimethoxysilane. The concave particles used in the compositions of the present invention thus advantageously have a crosslinked structure composed of SiO₂ units and of RSiO_{1.5} units where R is preferably either a methyl group or a hydroxyl group.

Use may independently be made, as hydrolysis and polymerization catalysts, of basic catalysts, such as sodium hydroxide, potassium hydroxide, sodium carbonate, sodium hydrogencarbonate, or amines (such as ammonia, trimethylamine, triethylamine or tetramethylammonium hydroxide), or acidic catalysts chosen from organic acids, such as citric acid, acetic acid, methanesulphonic acid, p-toluenesulphonic acid, dodecylbenzenesulphonic acid, dodecylsulphonic acid, or inorganic acids, such as hydrochloric acid, sulphuric acid, phosphoric acid. When it is present, the surfactant used is preferably a nonionic or anionic surfactant or a mixture of the two. Sodium dodecyl-benzenesulphonate can be used as anionic surfactant. The end of the hydrolysis is marked by the disappearance of the products (III) and (IV), which are insoluble in water, and the production of a homogeneous liquid layer.

The condensation stage (b) can use the same catalyst as the hydrolysis stage or another catalyst chosen from those especially mentioned above.

On conclusion of this process, a suspension in water of fine organosilicone particles is obtained, which particles can optionally be separated subsequently from their medium. The process described above can thus comprise an additional stage of filtration, for example on a membrane filter, of the product resulting from stage (b), optionally followed by a stage of centrifuging the filtrate, intended to separate the particles from the liquid medium, and then by a stage of drying the particles. Other separation methods can, of course, be employed.

The particles obtained preferably have a mean diameter ranging from 0.05 to 20 μm.

The shape of the portions of hollow spheres obtained according to the above process and their dimensions will depend in particular on the method used to bring the products into contact in stage (b).

A somewhat basic pH and introduction under cold conditions of the polymerization catalyst into the mixture resulting from stage (a) will result in portions of hollow spheres with the shape of round-bottomed “bowls”, whereas a somewhat acidic pH and dropwise introduction of the mixture resulting from stage (a) into the hot polymerization catalyst will result in portions of hollow spheres having a transverse cross section with the shape of a “horseshoe”.

According to a preferred embodiment of the invention, portions of hollow spheres with the shape of “bowls” are used. These can be obtained as disclosed in invention JP-2003 128 788.

Portions of hollow spheres with the shape of a horseshoe are also disclosed in invention JP-A-2000-191789.

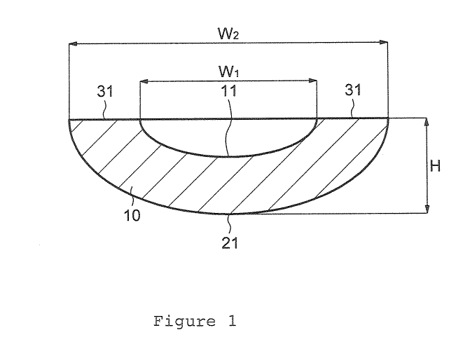

A concave particle formed of portions of spheres with the shape of a bowl is illustrated in transverse cross section in appended FIG. 1.

As emerges from this FIGURE, these concave portions are formed (in longitudinal cross section) of a small internal arc (11), of a large external arc (21) and of segments (31) which connect the ends of the respective arcs, the width (W1) between the two ends of the small internal arc (11) ranging from 0.01 to 8 μm, preferably from 0.02 to 6 μm, on average, the width (W2) between the two ends of the large external arc (21) ranging from 0.05 to 20 μm, preferably from 0.06 to 15 μm, on average, and the height (H) of the large external arc (21) ranging from 0.015 to 15 μm, preferably from 0.03 to 15 μm, on average.

The dimensions especially mentioned above are obtained by calculating the mean of the dimensions of one hundred particles chosen on an image obtained with a scanning electron microscope.

Mention may especially be made, as concave particles which can be used according to the invention, of:

particles composed of the crosslinked organosilicone TAK-110 (crosslinked methylsilanol/silicate polymer) from Takemoto Oil & Fat, with the shape of a bowl, with a width of 2.5 μm, a height of 1.2 μm and a thickness of 150 nm (particles sold under the name NLK-506 by Takemoto Oil & Fat); these particles will be preferably used in the compositions according to the present invention;

particles composed of the crosslinked organosilicone TAK-110 (crosslinked methylsilanol/silicate polymer) from Takemoto Oil & Fat, with the shape of a bowl, with a width of 2.5 μm, a height of 1.5 μm and a thickness of 350 nm;

particles composed of the crosslinked organosilicone TAK-110 (crosslinked methylsilanol/silicate polymer) from Takemoto Oil & Fat, with the shape of a bowl, with a width of 0.7 μm, a height of 0.35 μm and a thickness of 100 nm;

particles composed of the crosslinked organosilicone TAK-110 (crosslinked methylsilanol/silicate polymer) from Takemoto Oil & Fat, with the shape of a bowl, with a width of 7.5 μm, a height of 3.5 μm and a thickness of 200 nm.

Advantageously, the concave particles, in particular the portions of hollow spheres, are present in the composition according to the invention in a quantity ranging from 0.01% to 20% by weight, preferably ranging from 0.5% to 10% by weight and preferentially ranging from 0.5% to 5% by weight, with respect to the total weight of the composition.

“Dispersant for concave particles” which can be used in the compositions according to the present invention is understood to mean any compound capable of resulting in a homogeneous distribution of the concave particles in the medium of the composition.

The dispersants for concave particles which can be used in the compositions according to the present invention are generally water-soluble or fat-soluble polymers; advantageously, these dispersants are vinylpyrrolidone homopolymers and polyalkylene glycols with a molecular weight of less than 20 000 g/mol.

According to a particularly preferred form of the invention, the vinylpyrrolidone homopolymers which can be used in the compositions according to the present invention have a molar mass of less than 20 000 g/mol, preferably less than 10 000 g/mol.

Mention may especially be made, as vinylpyrrolidone homopolymers which can be used in the compositions according to the present invention, of the following polymers:

| | Number-average molar | |
|---|---|---|
| Commercial name | mass (g/mol) | Supplier |
| Luviskol K 17 powder | 2500 | BASF |
| Kollidon 17 PF | 2500 | BASF |
| Kollidon 12 PF | 2500 | BASF |
| Kollidon 12 PF | 10000 | BASF |

The polyalkylene glycols which can be used in the compositions according to the present invention are preferably chosen from those having an alkylene group comprising from 1 to 4 carbon atoms, in particular polyethylene glycols, polypropylene glycols and polybutylene glycols. These polyalkylene glycols exhibit a molecular weight of less than 20 000 g/mol, preferably of less than 10 000 g/mol. Mention may especially be made, by way of example, of the polyethylene glycol with a molecular weight of 1450 g/mol supplied by Union Carbide under the name “Sentry 10 Polyethylene Glycol”.

Advantageously, the dispersants for concave particles, in particular vinylpyrrolidone homopolymers and polyalkylene glycols with a molecular weight of less than 20 000 g/mol, are used in an amount ranging from 0.01% to 10%, preferably from 0.1% to 5% and more preferably still from 0.2% to 2.5% by weight, with respect to the total weight of the composition.

According to an advantageous specific embodiment, the compositions according to the present invention comprise porous fillers. The introduction of porous particles into a composition according to the present invention comprising concave particles and at least one dispersant for these concave particles makes it possible to enhance the mattifying nature of the composition or, for a given mattness value, the introduction of porous particles into a composition according to the present invention makes it possible to use a reduced amount of concave particles.

Within the meaning of the present invention, the term “porous filler” denotes any porous spherical or nonspherical particle which is insoluble in the compositions according to the present invention and which differs from the concave particles used in the compositions according to the present invention.

The porous fillers which can be used in the compositions of the present invention are solid particles which are insoluble, under standard temperature and pressure conditions, in the medium into which they are introduced and which, when they are added to an oil-in-water emulsion comprising 1.00% by weight of ammonium polyacryldimethyltauramide, 5.0% by weight of cyclohexasiloxane, 1.70% by weight of glycerol, 0.30% by weight of stearyl alcohol, 0.70% by weight of glyceryl stearate/PEG 100 stearate, 0.50% by weight of dimyristyl tartrate/cetearyl alcohol/C12-15 pareth-7/PPG25 laureth-25, 1.5% by weight of concave particles, 2% by weight of polyvinylpyrrolidone and 0.50% by weight of preservatives (the remainder to 100% by weight being water) with respect to the total weight of the composition, make it possible to reduce the R value of this composition; preferably, they make it possible to reduce the R value to a value of less than or equal to 1, more preferably still to a value of less than or equal to 0.75.

The porous fillers advantageously exhibit a mean size by volume of less than 40 μm and in particular of less than 30 μm, indeed even of less than 15 μm, including 35, 25, 20, 10, 5 and less than 5 μm, including all values and subranges between stated values. Generally, the composition according to the invention can comprise various fillers of mineral or organic origin. These fillers can be of any shape; in particular, they can be of plated, spherical or oblong shape, and the like. These fillers can be of any crystallographic form, in particular as sheets, cubic, hexagonal, orthorhombic, and the like. These fillers are porous; this porosity can in particular be reflected by a specific surface area of the particles of greater than 10 m²/g, and in particular of greater than 50 m²/g.

Preferably, the porous fillers are spherical.

According to another preferred form, these porous fillers are of inorganic nature.

More particularly, these fillers can, for example, be chosen from:

porous silica microparticles, such as, for example, Silica Beads SB150 and SB700 from Miyoshi, with a mean size of 5 microns; Sunspheres Series-H from Asahi Glass, such as, for example, Sunspheres H33, H51 and H53, with mean sizes of 3, 5 and 5 microns respectively;

powders formed of acrylic copolymers, in particular of poly(methyl(meth)acrylate) (PMMA), such as, for example, the hollow PMMA spheres sold under the name Covabead LH85 by Wacker;

composite talc/titanium dioxide/alumina/silica powders, such as those sold under the name Coverleaf AR-80 by Catalyst & Chemicals;

polyamide (for example Nylons®) powders, such as, for example, the Nylon 12 particles of the Orgasol type from Atofina, with a mean size of 10 microns;

and their mixtures.

The levels introduced depend on the effect desired.

Advantageously, the porous fillers are used in an amount ranging from 0.1% to 10%, preferably from 0.5% to 5% and more preferably still from 0.5% to 3% by weight, with respect to the total weight of the composition.

The composition in accordance with the invention can also comprise at least one cosmetic active principle.

The cosmetic active principles which can be used in the compositions according to the present invention include antioxidants, physical and chemical sunscreens, vitamins (in particular C, B3, PP, B5, E, K1 and their derivatives, such as their esters), moisturising active principles, such as polyols and in particular glycerol, self-tanning compounds, antiwrinkle active agents, ceramides, DHEA and its derivatives, coenzyme Q10, agents for combating pollution or free radicals, dermo-relaxing active principles, soothing agents, agents which stimulate the synthesis of dermal or epidermal macromolecules and/or which prevent their decomposition, antiglycation agents, agents which combat irritation, desquamating agents, NO-synthase inhibitors, agents which stimulate the proliferation of fibroblasts or keratinocytes and/or the differentiation of keratinocytes, agents which act on the microcirculation, agents which act on the energy metabolism of the cells, healing agents, and their mixtures.

Specifically, the compositions according to the present invention comprise an antiageing cosmetic active principle chosen from compounds which enhance collagen synthesis, compounds which inhibit the decomposition of collagen, dermo-decontracting agents, agents which inhibit the glycation of proteins, agents which enhance the proliferation of keratinocytes, agents which enhance the differentiation of keratinocytes, and their mixtures.

Specifically, the compositions according to the present invention comprise a whitening cosmetic active principle.

The whitening cosmetic active principles which can be used in the compositions according to the present invention include depigmenting, antipigmenting or propigmenting agents and their mixtures.

The depigmenting or antipigmenting agents capable of being incorporated in the composition according to the present invention comprise, for example, the following compounds: kojic acid; ellagic acid; arbutin and its derivatives, such as those disclosed in inventions EP-895 779 and EP-524 109; hydroquinone; aminophenol derivatives, such as those disclosed in inventions WO 99/10318 and WO 99/32077, in particular N-cholesteryloxycarbonyl-para-aminophenol and N-ethyloxycarbonyl-para-aminophenol; iminophenol derivatives, in particular those disclosed in invention WO 99/22707; L-2-oxothiazolidine-4-carboxylic acid or procysteine, and its salts and esters; calcium D-pantetheinesulphonate; ascorbic acid and its derivatives, in particular ascorbyl glucoside; and plant extracts, in particular of liquorice, of blackberry, of skullcap and of Bacopa monnieri, without this list being limiting.

Mention may especially be made, as propigmenting agent, of the extract of burnet (Sanguisorba officinalis) sold by Maruzen and chrysanthemum extracts (Chrysanthemum morifolium).

The antiageing active principles which can be used in the compositions according to the present invention include the following compounds:

compounds which enhance the synthesis of collagen, such as extracts of Centella asiatica; asiaticosides and derivatives; ascorbic acid or vitamin C and its derivatives; synthetic peptides, such as iamine, CL biopeptide or palmitoyloligopeptide, sold by Sederna; peptides extracted from plants, such as the soybean hydrolysate sold by Coletica under the trade name Phytokine®; and plant hormones, such as auxins and lignans and/or elastin such as the extract of Saccharomyces cerevisiae sold by LSN under the trade name Cytovitin®; and the extract of the alga Macrocystis pyrifera sold by Secma under the trade name Kelpadelie® and/or of glycosaminoglycans such as the product of fermentation of milk by Lactobacillus vulgaris sold by Brooks under the trade name Biomin yogourth®; the extract of the brown alga Padina pavonica sold by Alban Müller under the trade name HSP3®; and the extract of Saccharomyces cerevisiae available in particular from Silab under the trade name Firmalift® or from LSN under the trade name Cytovitin® and/or of proteglycans and/or of fibronectin, such as the extract of Salina zooplankton sold by Seporga under the trade name GP4G®; the yeast extract available in particular from Alban Müller under the trade name Drieline®; and the palmitoyl pentapeptide sold by Sederna under the trade name Matrixil® and/or of laminin;

the compounds which inhibit collagen decomposition (such as retinoids and derivatives; oligopeptides and lipopeptides; lipoamino acids; the malt extract sold by Coletica under the trade name Collalift®; extracts of blueberry or of rosemary; lycopene; or isoflavones, their derivatives or the plant extracts comprising them, in particular extracts of soybean, sold, for example, by Ichimaru Pharcos under the trade name Flavosterone SB®, of red clover, of flax, of kakkon or of sage and/or of elastin such as the peptide extract of Pisum sativum seeds sold by LSN under the trade name Parelastyl®; heparinoids; and pseudodipeptides such as {2-[acetyl(3-trifluoromethylphenyl)amino]-3-methylbutyrylamino}acetic acid;

dermo-relaxing agents, such as alverine and its salts, manganese salts and in particular manganese gluconate, magnesium salts and in particular magnesium gluconate and sulphate, the hexapeptide Argireline R sold by Lipotec, adenosine, and also sapogenins and the natural extracts, in particular of Dioscorea opposita or of Dioscorea villosa (wild yam), comprising them, and also Boswellia serrata extracts;

agents which inhibit the glycation of proteins, such as plant extracts of the Ericaceae family, in particular an extract of blueberry (Vaccinium angustifolium); ergothioneine and its derivatives; and hydroxystilbenes and their derivatives, such as resveratrol and 3,3′,5,5′-tetrahydroxystilbene;

agents which enhance the proliferation of keratinocytes (such as retinoids including retinol and retinyl palmitate, adenosine, phloroglucinol, the extracts of walnut meal sold by Gattefosse and the extracts of Solanum tuberosum sold by Sederma) and/or of fibroblasts (such as plant proteins or polypeptides, extracts, in particular of soybean, and plant hormones, such as gibberellins and cytokinins);

agents which enhance the differentiation of keratinocytes, such as inorganic materials, including calcium; a peptide extract of lupin, such as that sold by Silab under the trade name Structurine®; sodium beta-sitosteryl sulphate, such as that sold by Seporga under the trade name Phytocohesine®; and a water-soluble extract of maize, such as that sold by Solabia under the trade name Phytovityl®; a peptide extract of Voandzeia substerranea, such as that sold by Laboratoires Sérobiologiques under the trade name Filladyn LS 9397®; and lignans, such as secoisolariciresinol; and their mixtures.

Specifically, the compositions according to the present invention comprise an active principle for the cosmetic treatment of greasy skin.

The active principles for the cosmetic treatment of greasy skin include zinc salts and in particular zinc gluconate; antibacterials, such as salicylic acid, triclosan, lipacid, clove extract, octopirox, hexamidine or antiacne active principles.

The composition according to the invention preferably comprises from 0.001 to 10%, more preferably from 0.01 to 5%, by weight of at least one active principle, with respect to the total weight of the composition.

The composition in accordance with the invention can also comprise at least one cosmetic additive chosen from dyes, pigments, fragrances, tightening agents, preservatives, sequestering agents, fat-soluble or water-soluble active principles, pH adjusters (acids or bases), hydrophilic or lipophilic gelling and/or thickening agents, emollients, film-forming agents, surfactants and their mixtures.

The cosmetic additives are generally present in an amount of, for each of them, between 0.01 and 20% by weight, with respect to the weight of the composition.

Of course, a person skilled in the art will take care to choose this or these optional additional compounds so that the advantageous properties intrinsically attached to the composition in accordance with the invention are not, or not substantially, detrimentally affected by the envisaged addition or additions.

The compositions described in the present invention can be provided in any form, including the form of direct, inverse or multiple (water-in-oil-in-water or else oil-in-water-in-oil) emulsions, of cream gels, of lotions, of sticks, of sprays or of anhydrous compositions.

Since the composition according to the invention is preferably intended for topical application, it preferably contains a physiologically acceptable medium, i.e. a medium compatible with the skin, the mucous membranes, the hair and the scalp. The composition may be especially a cosmetic or dermatological composition. For example, it may be an aqueous medium that consists of water or of a mixture of water and of at least one cosmetically or dermatologically acceptable solvent chosen for example from C1-C4 lower alcohols, such as ethanol, isopropanol, tert-butanol or n-butanol; polyols such as glycerol, propylene glycol and polyethylene glycols; and mixtures thereof.

The examples which follow serve to illustrate the invention without, however, exhibiting a limiting nature.

### EXAMPLES

I. Preparation of the Compositions

The following compositions were prepared.

The amounts below are given in percentage by weight with respect to the total weight of the composition.

| | 1 | | | 4 |
|---|---|---|---|---|
| | (compara- | | | (compara- |
| Example | tive) | 2 | 3 | tive) |
| Ammonium | 1.00% | 1.00% | 1.00% | 1.00% |
| polyacryldimethyl- | | | | |
| tauramide (1) | | | | |
| Cyclohexasiloxane | 5.0% | 5.0% | 5.0% | 5.0% |
| Glycerol | 1.70% | 1.70% | 1.70% | 1.70% |
| Stearyl alcohol | 0.30% | 0.30% | 0.30% | 0.30% |
| Glyceryl stearate/ | 0.70% | 0.70% | 0.70% | 0.70% |
| PEG 100 stearate | | | | |
| Dimyristyl tartrate/ | 0.50% | 0.50% | 0.50% | 0.50% |
| cetearyl alcohol/ | | | | |
| C12-15 pareth- | | | | |
| 7/PPG25 laureth-25 | | | | |
| Concave | 1.50% | 1.50% | 1.50% | — |
| organopolysiloxane | | | | |
| particles (2) | | | | |
| Polyvinylpyrrolidone | — | 2% | 2% | 2% |
| Porous particles (3) | — | — | 1.50% | 1.50% |
| Preservative | 0.50% | 0.50% | 0.50% | 0.50% |
| Water | qs for | qs for | qs for | qs for |
| | 100 | 100 | 100 | 100 |

(1): Hostacerin AMPS from Clariant
(2): NLK-506 from Takemoto Oil & Fat
(3): Porous silica SB700 particles from Miyoshi

The compositions of the following examples are prepared by adding, with stirring, the oily phase, heated to approximately 65° C., to the aqueous phase, heated to the same temperature.

II. Mattness Test

The mattness of the compositions of Examples 1 to 4 was measured in accordance with the protocol set out above, which makes use of a measurement with a gonioreflectometer on a contrast card (Prufkarte type 24/5-250 cm2 sold by Erichsen). The result obtained is the ratio R of the specular reflection to the diffuse reflection. The R value decreases as the mattifying effect increases.

| Compo- | Example 1 | | | Example 4 |
|---|---|---|---|---|
| sition | (comparative) | Example 2 | Example 3 | (comparative) |
| R | 1.30 + 0.10 | 0.92 + 0.03 | 0.67 + 0.01 | 1.85 + 0.05 |

These in vitro results show that, on combining the concave organopolysiloxane particles with a vinylpyrrolidone homopolymer (Examples 2 and 3), a better mattness result is obtained than is obtained with the comparative compositions. Furthermore, the use of a composition formed of concave organopolysiloxane particles with a vinylpyrrolidone homopolymer and porous fillers (Example 3) makes it possible to obtain better mattifying properties. In comparison, the combination of porous silica fillers with a vinylpyrrolidone homopolymer does not make it possible to achieve satisfactory results.

III Sensory Evaluation:

The mattifying effect and the cosmetic aspects of the composition of Example 3 were evaluated on a panel of 7 women having greasy skin. For these women exhibiting shiny skin, the composition of Example 3 has the effect of mattifying and of rendering the texture of the skin finer, the pores appear less dilated and the complexion more unified.

The above written description of the invention provides a manner and process of making and using it such that any person skilled in this art is enabled to make and use the same, this enablement being provided in particular for the subject matter of the appended claims, which make up a part of the original description and including a nonpulverulent composition wherein it comprises concave particles and at least one dispersant for these concave particles.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

The invention method and composition is preferably used by subjects desirous of the benefits noted herein, subjects “in need of” these benefits. Such subjects are typically desirious of cosmetically treating, caring for or making up keratinous substances, in particular the skin, of conferring a matt appearance on keratinous substances, for softening imperfections of the relief of the skin and/or for concealing microrelief features, wrinkles, fine lines and pores of the skin. Naturally, one using the invention as disclosed will use an amount of the invention composition effective to provide such benefits. Such amount is inclusive of an amount of the compositions described herein at the disclosed concentrations of necessary ingredients sufficient to cover the area of the, e.g., skin being treated in a single application, and of course includes that amount applied upon repeated application, for example on a daily basis over a course of days, weeks, etc. In a preferred embodiment the invention process includes multiple applications of the invention composition to the area(s) of skin in need of attention.

1. A nonpulverulent composition comprising, in a physiologically acceptable medium, concave particles and at least one dispersant for the concave particles.

2. The composition according to claim 1, wherein said at least one dispersant is chosen from vinylpyrrolidone homopolymers and polyalkylene glycols with a molecular weight of less than 20,000 g/mol, and mixtures thereof.

3. The composition according to claim 1, wherein the said concave particles have a mean diameter ranging from 0.05 μm to 20 μm.

4. The composition according to claim 1, wherein the concave particles are in the form of portions of hollow spheres exhibiting a transverse cross section with the shape of a horseshoe or arch.

5. The composition according to claim 1, wherein the concave particles comprise an organosilicone material.

6. The composition according to claim 5, wherein the organosilicone material is a crosslinked polysiloxane with a three-dimensional structure comprising units of formula (I): SiO2, and of formula (II): R1SiO1.5,
in which R1 denotes an organic group having a carbon atom directly connected to the silicon atom.

7. The composition according to claim 6, wherein the organic group is an unreactive organic group.

8. The composition according to claim 7, wherein the unreactive organic group is a C1-C4 alkyl group or a phenyl group.

9. The composition according to claim 7, wherein the unreactive organic group is a methyl group.

10. The composition according to claim 6, wherein the organic group is a reactive group chosen from an epoxy group, a (meth)acryloxy group, an alkenyl group, a mercaptoalkyl, aminoalkyl or haloalkyl group, a glyceroxy group, a ureido group and a cyano group.

11. The composition according to claim 6, wherein the organic group is a reactive group chosen from an epoxy group, a (meth)acryloxy group, an alkenyl group, a mercaptoalkyl group and a aminoalkyl group.

12. The composition according to claim 6, wherein the organosilicone material comprises the units (I) and (II) according to a unit (I)/unit (II) molar ratio ranging from 30/70 to 50/50.

13. The composition according to claim 6, wherein the organosilicone particles are obtained according to a process comprising:
(a) the introduction into an aqueous medium, in the presence of at least one hydrolysis catalyst and optionally of at least one surfactant, of a compound (III) of formula SiX4 and of a compound (IV) of formula RSiY3, where X and Y denote, independently of one another, a C1-C4 alkoxy group, an alkoxyethoxy group including a C1-C4 alkoxy group, a C2-C4 acyloxy group, an N,N-dialkylamino group including a C1-C4 alkyl group, a hydroxyl group, a halogen atom or a hydrogen atom and R denotes an organic group comprising a carbon atom connected directly to the silicon atom to form a mixture; and
(b) an operation in which the mixture is brought into contact with an aqueous solution including at least one polymerization catalyst and optionally at least one surfactant, at a temperature of between 30 and 85° C., for at least two hours.

14. The composition according to claim 13, wherein, in stage (a), the molar ratio of the compound (III) to the compound (IV) ranges from 30/70 to 50/50.

15. The composition according to claim 13, wherein the ratio by weight of the water to the total of the compounds (III) and (IV) ranges from 10/90 to 70/30.

16. The composition according to claim 13, wherein the organic group is a reactive organic group.

17. The composition according to claim 13, wherein the organic group is an unreactive organic group chosen from a C1-C4 alkyl group and a phenyl group.

18. The composition according to claim 17, wherein the organic unreactive group is a methyl group.

19. The composition according to claim 13, wherein the organic group is a reactive organic group chosen from an epoxy group, a (meth)acryloyloxy group, an alkenyl group, a mercaptoalkyl group, an aminoalkyl group, a haloalkyl group, a glyceroxy group, a ureido group and a cyano group.

20. The composition according to claim 13, wherein the organic group is a reactive organic group chosen from an epoxy group, a (meth)acryloyloxy group, an alkenyl group, a mercaptoalkyl group and an aminoalkyl group.

21. The composition according to claim 13, wherein the hydrolysis and polymerization catalysts are chosen independently from sodium hydroxide, potassium hydroxide, sodium carbonate, sodium hydrogencarbonate, ammonia, trimethylamine, triethylamine, tetramethylammonium hydroxide, citric acid, acetic acid, methanesulphonic acid, p-toluenesulphonic acid, dodecylbenzenesulphonic acid, dodecylsulphonic acid, hydrochloric acid, sulphuric acid and phosphoric acid.

22. The composition according to claim 1, wherein the concave particles are formed in longitudinal cross section of a small internal arc, of a large external arc, and of segments which connect the ends of the respective arcs, the width between the two ends of the small internal arc ranging from 0.01 to 8 μm, on average, the width between the two ends of the large external arc ranging from 0.05 to 20 μm, on average, the height of the large external arc ranging from 0.015 to 15 μm on average.

23. The composition according to claim 4, wherein the portions of hollow spheres are present in a quantity ranging from 0.01% to 20% by weight with respect to the total weight of the composition.

24. The composition according to claim 23, wherein the dispersant is a vinylpyrrolidone homopolymer with a molecular weight of less than 20 000 g/mol.

25. The composition according to claim 1, wherein the dispersant is chosen from polyethylene glycols, polypropylene glycols, polybutylene glycols and mixtures thereof.

26. The composition according to claim 1, wherein the amount of dispersant ranges from 0.01% to 10% by weight with respect to the total weight of the composition.

27. The composition according to claim 1, further comprising at least one porous filler which is insoluble under standard temperature and pressure conditions and which, when added to an oil-in-water emulsion comprising 1.00% by weight of ammonium polyacryldimethyltauramide, 5.0% by weight of cyclohexasiloxane, 1.70% by weight of glycerol, 0.30% by weight of stearyl alcohol, 0.70% by weight of glyceryl stearate/PEG 100 stearate, 0.50% by weight of dimyristyl tartrate/cetearyl alcohol/C12-15 pareth-7/PPG25 laureth-25, 1.5% by weight of concave particles, 2% by weight of polyvinylpyrrolidone and 0.50% by weight of preservatives, it reduces the R value of this composition, the said R value being the ratio of the specular reflection to the diffuse reflection.

28. The composition according to claim 27, wherein the at least one porous filler is chosen from porous silica microparticles, powders formed of acrylic copolymers, composite talc/titanium dioxide/alumina/silica powders, polyamide powders and mixtures thereof.

29. The composition according to claim 27, wherein the amount of porous filler ranges from 0.1% to 10% with respect to the total weight of the composition.

30. The composition according to claim 1, further comprising at least one cosmetically active principle.

31. The composition according to claim 30, wherein the cosmetically active principle is at least one of an antiageing cosmetic active principle chosen from compounds which enhance collagen synthesis, compounds which inhibit the decomposition of collagen, dermo-decontracting agents, agents which inhibit the glycation of proteins, agents which enhance the proliferation of keratinocytes, agents which enhance the differentiation of keratinocytes; a whitening cosmetic active principle chosen from depigmenting, antipigmenting or propigmenting agents and mixture thereof; active principles for the cosmetic treatment of greasy skin, antibacterials or antiacne active principles, antioxidants, physical and chemical sunscreens, vitamins, moisturising active principles, self-tanning compounds, antiwrinkle active agents, ceramides, DHEA and its derivatives, coenzyme Q10, agents for combating pollution or free radicals, dermo-relaxing active principles, soothing agents, agents which stimulate the synthesis of dermal or epidermal macromolecules and/or which prevent their decomposition, antiglycation agents, agents which combat irritation, desquamating agents, NO-synthase inhibitors, agents which stimulate the proliferation of fibroblasts or keratinocytes and/or the differentiation of keratinocytes, agents which act on the microcirculation, agents which act on the energy metabolism of the cells, healing agents, and their mixtures.

32. The composition according to claim 30, wherein it comprises from 0.001 to 10% by weight of at least one cosmetically active principle with respect to the total weight of the composition.

33. The composition according to claim 1, further comprising at least one cosmetic additive chosen from dyes, pigments, fragrances, tightening agents, preservatives, sequestering agents, fat-soluble or water-soluble active principles, pH adjusters, hydrophilic or lipophilic gelling agents, hydrophilic or lipophilic thickening agents, emollients, film-forming agents, surfactants and their mixtures.

34. The composition according to claim 1, wherein said composition is in the form of direct, inverse or multiple emulsion, a cream gel, a lotion, a stick, a spray or of an anhydrous composition.

35. A process for cosmetically treating, caring for making up keratinous substances, in particular the skin, comprising applying to a keratinous substance the composition according to claim 1.

36. The process of claim 35, where said process is one for conferring a matt appearance on a keratinous substance, for softening imperfections of the relief of the skin and/or for concealing microrelief features, wrinkles, fine lines and/or pores of the skin, comprising applying an effective amount of a composition comprising, in a physiologically acceptable medium, concave particles and at least one dispersant for the concave particles to skin in need thereof.
